(19) United States
(12) Patent Application Publication
(10) Pub. No.: US 2004/0065571 A1
(43) Pub. Date: Apr. 8, 2004

(54) Sharps protector and sterilization insert

(75) Inventor: Clifford Eugene Gammons, Loudon, TN (US)

Correspondence Address: PITTS AND BRITTIAN P C, P O BOX 51295, KNOXVILLE, TN, 37950-1295, US

(73) Assignee: Adroit Medical Systems, Inc., Loudon, TN

(21) Appl. No.: 10/264,396

(22) Filed: Oct. 4, 2002

(51) Int. Cl. A01N 1/00; A61L 2/00

(52) U.S. Cl. 206/363; 422/28; 422/297; 422/300

(57) ABSTRACT

A surgical instrument holder for positioning of a surgical tool on the holder for sterilization and containment of sharp ends, The holder includes a first end having a first biased flap for containment of a first end of the tool. A second end of the holder includes a second biased flap for containment of a second end of the tool with each biased flap being folded onto the tool when positioned therebetween to limit longitudinal movement of the tool. A retainer clip is positioned between the first end and the second end and is adapted to receive the surgical tool therethrough to limit the lateral movement of the tool. The first biased flap includes a flap extension for positioning against the surgical tool for biasing the tool in a partially open position during sterilization. A method of preparation of a surgical tool for sterilization is also disclosed.

### CROSS REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention

This invention pertains to a holder for containing sharp surgical tools. More particularly, this invention pertains to a holder for positioning sharp surgical tools for sterilization.

2. Description of the Related Art

In both the fields of medical research and medical treatment, and particularly in the surgical arts, it is well known that maintaining the integrity of a sterile field for each surgical tool introduced into the operating room is of paramount importance. In practice this has meant that many items intended for use in a surgical field are double packaged with a completely sealed outer packaging element surrounding an inner packaging element in or on which the sterilizable item is located. At some point in the production cycle the double-packaged item is sterilized so that when the item arrives at the point of use, both the item and the inner packaging element are sterile. If sharp objects such as surgical tools, including scissors, knives, rods and forceps, are not restricted from movement within the inner packaging element, sharp edges or ends will protrude from the inner packaging element during shipment with disruption of the sterility of the surgical tool.

Further, it is preferred that the packaging element positions the surgical tool in a configuration during autoclaving for sterilization of all surfaces of the surgical tool, and provides a semi-rigid support for maintaining the surgical tool in an easily removable position when selected for use in a surgical procedure. Accordingly, there is a need for a holder for positioning an autoclavable instrument thereon for sterilization and for introduction into a sterile field without disruption of the sterility of the instrument.

It is therefore an object of the present invention to provide a holder of a surgical instrument for positioning and maintaining the surgical instrument in a partially opened position for sterilization and sterile storage of the surgical instrument.

An additional object of the present invention is to provide a surgical tool holder that contains the sharp ends of the tool while maintaining the tool in a partially opened position for ease of separation from the holder during a surgical procedure.

Other objects and advantages over the prior art will become apparent to those skilled in the art upon reading the detailed description together with the drawings and claims as provided herein.

### BRIEF SUMMARY OF THE INVENTION

The present invention includes a holder for positioning of a sharp instrument such as a surgical tool thereon for autoclaving and transfer for use in a medical procedure. The holder includes a first surface for positioning the surgical tool thereon and includes a first end and a second end. Each first and second end is adapted to be folded as flaps onto the first surface, with each of the first and second ends being folded over an end portion of a surgical tool positioned therebetween on the first surface of the holder. At least one of the first and second ends includes a biased flap extension foldable toward and insertable against the surgical tool for maintaining the surgical tool in a partially opened position. A retainer clip is disposed along a midline of the first surface of the holder. The retainer clip is configured to limit the lateral movement of the surgical tool in conjunction with the folded flaps limiting the longitudinal movement of the surgical tool. The retainer clip is formed between an anterior opening and a posterior opening in the first surface of the holder. The anterior and posterior openings are sized to accept therethrough the surgical tool. The holder is composed of semi-rigid material and is autoclavable without deterioration in rigidity. The holder is configured in a substantially planar shape in one embodiment, may be sized in a variety of lengths and shapes, and is insertable into a pliable, nonwoven, and autoclavable sleeve of material A method of preparation of a surgical tool for sterilization is disclosed including steps of mounting a surgical tool on a holder, inserting the surgical tool in a partially open position into an autoclavable sleeve, and sterilizing the holder, the surgical tool, and the sleeve for a period of time determined by an operator for adequate sterilization.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a top view of a holder of the present invention having opened end flaps with a partially open surgical tool positioned therebetween;

FIG. 2 is a side view of the holder of FIG. 1, illustrating the surgical instrument contained between end flaps folded against the instrument;

FIG. 3 is a top view of an alternative embodiment of the holder of the present invention having a surgical implant instrument positioned thereon and having the holder partially inserted into an autoclavable sleeve; and

FIG. 4 is a side view of the holder of FIG. 3, illustrating the surgical implant instrument contained between the folded end flaps and having the holder partially inserted into an autoclavable sleeve.

### DETAILED DESCRIPTION OF THE INVENTION

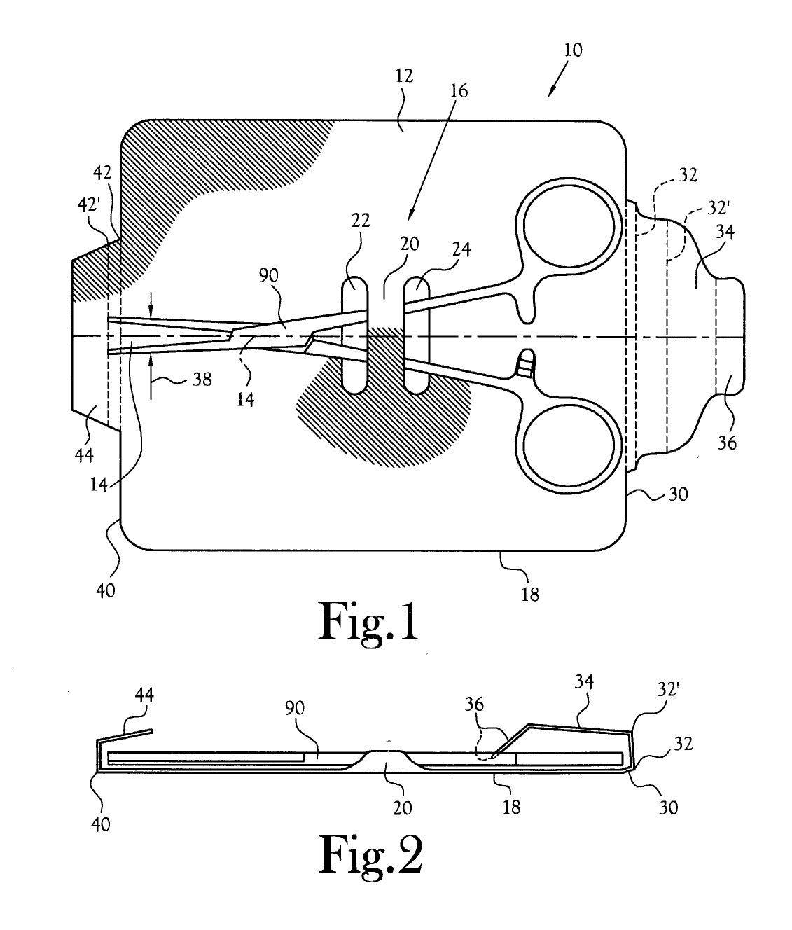

An embodiment of the present invention includes a holder 10 as illustrated in FIG. 1, for positioning of a sharp instrument such as a surgical tool 90 thereon for autoclaving and transfer to an operating room. The holder 10 includes a first surface 12 for positioning the surgical tool 90 thereon and includes a base surface 18 composed of a semi-rigid material so as to maintain a substantially planar configuration for the base surface 18. The holder 10 includes a midline 14 along the length axis of the first surface 12 on which a mid-portion 16 segment is separated by at least two openings 22, 24 in the mid-portion 16 segment. The two openings 22, 24 illustrated in FIGS. 1 and 2, form a loop 20 of material of the first surface 12, under which the surgical tool 90 may be removably positioned. An additional embodiment of holder 10 may include a second loop (not shown) proximate either end of the first surface 12.

The holder 10 includes a first end 30 and an opposed second end 40. The first end 30 is adapted to be folded at a first fold 32 and a second fold 32′, to form a first flap extension 34 that is manipulated by a person assembling the holder 10 to fold an articulated first end 36 proximate to the first surface 12. The second end 40 also includes a first fold 42 and a second fold 42′ to form a second flap extension 44 which extends along a portion of one end of the surgical tool for enclosure of the tool end. The first flap extension 34 and the second flap extension 44 are separately positioned toward each other when the surgical tool 90 is positioned therebetween to enclose each rounded and/or sharp end of the surgical tool 90 when positioned on the holder 10. In an alternative embodiment illustrated in FIG. 2, a second articulated end 46 is extended from the second flap extension 44, for insertion between the working surfaces of the surgical tool 90. When there is a need for the surgical tool during a medical procedure, each flap extension 34, 44 are separately manipulated with one or both hands to quickly remove the surgical tool 90 from the holder 10.

The first flap extension 34 includes a separator edge 36 that is biased toward and positionable against or between a portion of the handle, or the opposed end, of the surgical tool 90 for maintaining the tool in a partially open position. The insertion of the separator edge 36 between one end portion of the surgical tool 90, such as between the finger loops of a forceps, provides for a separation distance 38 to be maintained between the working surfaces of the opposed end of a tool such as forceps. A benefit exists, as recognized by those skilled in the art, to maintain a separation distance 38 between the working surfaces during a sterilization process, thereby ensuring all outer surfaces of the surgical tool 90 are sterilized.

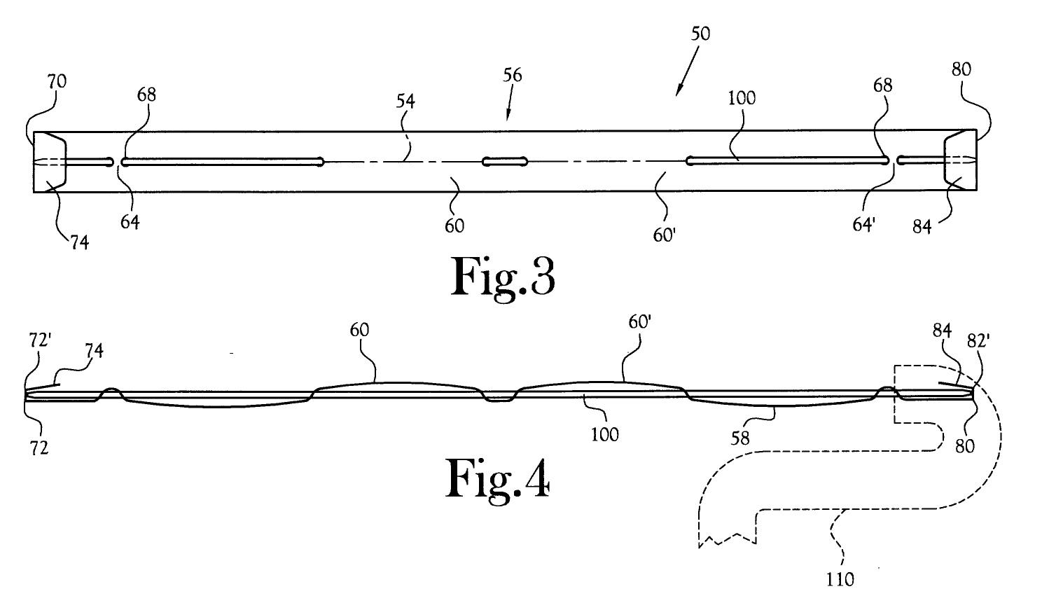

A retainer clip 20 is disposed along a midline 14 of the holder 10 at about a mid-portion 16 between the first end 30 and second end 40. The retainer clip 20 is configured to contain a middle portion of the surgical tool 90 when inserted under the retainer clip 20, which serves to limit the lateral movement of a tool retained therein. The retainer clip 20 is pliable and functions in concert with the first flap extension 34 and second flap extension 44 to limit the forward and rearward or longitudinal movement of the surgical tool 90 during preparation for autoclaving and during transport before use in a medical procedure. The retainer clip 20 is bounded by an anterior opening 22 and a posterior opening 24 in the first surface 12 of the holder 10. The anterior opening 22 and posterior opening 24 are oriented along the mid-portion 16 of the first surface 12 and are each sized in width to accept therethrough a middle portion of the surgical tool 90 (see FIGS. 1 and 2). An alternative embodiment of an elongated holder 50 includes a substantially planar shape having a plurality of retainer portions, is illustrated in FIGS. 3 and 4.

Either of the holders 10, 50 are configured in a substantially elongated shape and are sized to be insertable into a pliable, nonwoven, and autoclavable sleeve 110 of material. Each holder 10, 50 is composed of semi-rigid material such as high-density plastic that is autoclavable without deterioration in shape, and is pliable to allow bending of the middle segments and ends of the holder for containment of a surgical tool 10 or 100. The alternative embodiment of an elongated holder 50 is sized for support of an elongated surgical instrument 100 as illustrated in FIGS. 3 and 4. Elongated holder 50 includes a first surface 52 and a base surface 58 which are substantially planar, having a lengthwise midline 54 along which a plurality of holes 62, 62′, 66, 66′ are aligned. On anterior and posterior portions of a middle segment 56, are respective pairs of holes 62, 62′ and 66, 66′ through which an elongated surgical instrument 100 is insertable. Along the midline 54 of the first surface 52 is at least two retainer portions 60, 64 that are formed by the elongated surgical instrument being woven through each respective pair of holes and under respective portions of the pliable first surface 52. The plurality of retainer portions 60, 64 may include additional retainer portions 60′, 64′ spaced apart at opposed ends of the first surface 52 if a substantially elongated surgical instrument 100 is positioned on the first surface 52 through respective pairs of holes 62, 62′ and 66, 66′.

Elongated holder 50 includes a first end 70 that is adapted to be folded at a first fold 72 and second fold 72′, to form a first flap extension 74 that is manipulated by a person assembling the holder 50 to fold an articulated first end 76 proximate to the first surface 52. The second end 80 also includes a first fold 82 and a second fold 82′ to form a second flap extension 84. Each flap extension 74, 84 extends to enclose a portion of each respective end of the surgical instrument 100. An alternative embodiment of the elongated holder 50 includes the first surface 52 having a longitudinal midline from which the first surface curves upon itself (not shown), with a longitudinal opening for insertion of a surgical tool 90 or a surgical instrument 100 within the interior of the tubular shape. The surgical tool 90 or surgical instrument 100 is maintained within the tubular shape by each flap extension 74, 84 being biased to enclose a portion of each respective end of the surgical tool 90 or surgical instrument 100.

A method of sterilization is disclosed including steps of mounting a surgical tool on a holder, with the tool positioned in a partially opened orientation so that the working surfaces of the tool are not clasped against each other. A step of inserting includes inserting the surgical tool mounted on the holder into an autoclavable sleeve. The sleeve is preferably composed of pliable, nonwoven and autoclavable material. A step of sterilizing includes the holder, the surgical tool, and the sleeve for a period of time determined by an operator.

From the foregoing description, it will be recognized by those skilled in the art that a surgical tool protector is disclosed that provides a semi-rigid holder that includes dual flaps and holder clips for positioning the surgical tool in a partially open position to allow each surface of the surgical tool to be adequately sterilized during autoclaving.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

Having thus described the aforementioned invention, we claim:

1. A surgical instrument holder for positioning of a surgical tool on said holder for sterilization comprising:
a holder having a first surface for positioning a surgical tool thereon, said holder including a first end and a second end, said first end having a first biased flap for containment of a first end of the surgical tool, said second end having a second biased flap for containment of a second end of the surgical tool, said first and second biased flaps being folded onto the surgical tool when positioned therebetween to limit the longitudinal movement of the surgical tool; and
a retainer clip positioned on said holder between said first end and said second end, said retainer clip adapted to receive the surgical tool therethrough, said retainer clip configured to limit the lateral movement of the surgical tool.

2. The surgical tool holder of claim 1 wherein said first biased flap of said first end including a biased flap extension for manipulation against the surgical tool for biasing the first end of the surgical tool in a partially open position.

3. The surgical tool holder of claim 1 wherein said second biased flap of said second end including a biased flap extension for manipulation against the surgical tool for biasing the second end of the surgical tool in a partially open position.

4. The surgical tool holder of claim 1 wherein said first surface including an anterior opening and a posterior opening through said first surface, said anterior opening and said posterior opening being disposed to respectively delineate an anterior side and a posterior side of said retainer clip, said anterior opening and said posterior opening being sized to accept the surgical tool therethrough.

5. The surgical tool holder of claim 1 wherein said holder is composed of semi-rigid material and is autoclavable without deterioration in rigidity.

6. The surgical tool holder of claim 2 wherein said biased flap extension of said first biased flap further including a flap extension having an extension width of lesser width that a width of said first biased flap, whereby said biased flap extension is removably insertable against an end segment of the surgical tool to maintain the surgical tool in said partially open position.

7. The surgical tool holder of claim 1 further comprising a sleeve of autoclavable material being substantially tubular and having a preselected length, said sleeve for receiving therein said holder having the surgical tool mounted thereon for sterilization.

8. A surgical instrument holder for positioning thereon a surgical tool for sterilization comprising:
a holder having a first surface for positioning the surgical tool thereon;
a first end of said first surface having a first flap end biased to be foldable upon said first surface;
a second end of said first surface opposed from said first end, said second end having a second flap end biased to be foldable upon said first surface, said first flap end and said second flap end being biased to contain therebetween each end of the surgical tool to limit the longitudinal movement of the surgical tool;
said first flap end having a flap extension of a width narrower than said first flap end, said flap extension being in biased engagement against a first end of the surgical tool to bias the surgical tool in a partially open position; and
a retainer clip positioned on said holder between said first end and said second end, said retainer clip having an anterior opening and a posterior opening disposed adjacent said retainer clip, said retainer clip adapted to receive the surgical tool therethrough to limit the lateral movement of the surgical tool.

9. The surgical instrument holder of claim 8 wherein said first flap end and said second flap end being of a length to enclose each end of the surgical tool, each of said first flap and said second flap end being manipulated to an extended position to slidably remove the surgical tool from said retainer clip for use of the surgical tool in a medical procedure.

10. The surgical instrument holder of claim 8 wherein said anterior opening and said posterior opening being disposed to respectively delineate an anterior side and a posterior side of said retainer clip, said anterior opening and said posterior opening being sized to accept the surgical tool therethrough.

11. The surgical instrument holder of claim 8 wherein said holder is composed of semi-rigid material and is autoclavable without deterioration in rigidity.

12. The surgical instrument holder of claim 8 further comprising a sleeve of autoclavable material being substantially tubular and having a preselected length, said sleeve for receiving therein said holder having the surgical tool mounted thereon for sterilization.

13. A surgical instrument holder for positioning of a surgical tool on said holder for sterilization comprising:
a holder having a first surface for positioning a surgical tool thereon, said holder including a first end and a second end, said first end having a first biased flap for containment of a first end of the surgical tool, said second end having a second biased flap for containment of a second end of the surgical tool, said first and second biased flaps being folded onto the surgical tool when positioned therebetween to limit the longitudinal movement of the surgical tool; and
a retainer clip positioned on said holder between said first end and said second end, said retainer clip adapted to receive the surgical tool therethrough, said retainer clip configured to limit the lateral movement of the surgical tool.

14. The surgical instrument holder of claim 13 wherein said first biased flap including a biased flap extension having an extension width of lesser width that a width of said first biased flap, whereby said biased flap extension is removably disposed against an end segment of the surgical tool to maintain the surgical tool in said partially open position.

15. The surgical instrument holder of claim 13 further comprising a sleeve of autoclavable material being substantially tubular and having a preselected length, said sleeve for receiving therein said holder having the surgical tool mounted thereon for sterilization.

16. The surgical instrument holder of claim 13 wherein said retainer clip includes a plurality of retainer clips disposed along a longitudinal midline of said holder, the surgical tool insertable through each respective retainer clip with each respective end of the surgical tool enclosed by said first biased flap and said second biased flap for containment of the surgical tool for sterilization and delivery for use in a medical procedure.

17. A method of preparation of a surgical tool for sterilization comprising the steps of:
mounting a surgical tool on a holder including a pair of opposed end flaps biased against each respective end of the surgical tool between said pair of end flaps;
positioning an end flap extension against one of the ends of the surgical tool thereby maintaining the surgical tool in a partially open position;
inserting the surgical tool maintained in said partially open position on the holder into a tubular sleeve; and
sterilizing said holder, the surgical tool, and said tubular sleeve for a period of time determined by an operator for adequate sterilization.
